(19) United States
(12) Patent Application Publication
(10) Pub. No.: US 2024/0423619 A1
(43) Pub. Date: Dec. 26, 2024

(54) SURGICAL ADJUNCTS INCLUDING FLUID BARRIERS

(71) Applicant: CILAG GMBH INTERNATIONAL, Zug (CH)

(72) Inventors: PEYTON HOPSON, Cincinnati, OH (US); Madelaine Franzoni, Cincinnati, OH (US); Michael J. Vendely, Lebanon, OH (US); Brody Frost, Fairfield, OH (US)

(21) Appl. No.: 18/485,145

(22) Filed: Oct. 11, 2023

Related U.S. Application Data
(60) Provisional application No. 63/522,660, filed on Jun. 22, 2023.

(51) Int. Cl. A61B 17/072; A61L 31/06; A61L 31/10; A61L 31/14

(52) U.S. Cl. CPC A61B 17/07292; A61L 31/06; A61L 31/10; A61L 31/146; A61L 2400/12

(57) ABSTRACT

The disclosed technology includes a surgical adjunct 604 that includes a polyurethane foam comprising a volumetric ratio of the polyurethane foam to the total volume of the surgical adjunct 604 is in a range of about 0.125 to about 0.325, and at least one hydrophobicity additive comprising at least one ceramic nanoparticle, at least one fatty acid, at least one ionic liquid, at least one long chain surfactant, at least one enteric coating, at least one photocurable resin, or combinations thereof.

### CROSS-REFERENCE TO RELATED APPLICATIONS

This application claims priority under 35 U.S.C. § 119 to U.S. Provisional Patent Application No. 63/522,660, filed Jun. 22, 2023, the entire contents of which are fully incorporated herein by reference.

### FIELD

The present invention relates generally to compressible surgical adjuncts, cartridges, cartridge assemblies and methods of making adjuncts and cartridge assemblies.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and is often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

### SUMMARY

There is provided, in accordance with an example of the present invention, a surgical adjunct, including a polyurethane foam comprising a volumetric ratio of the polyurethane foam to the total volume of the surgical adjunct is in a range of about 0.125 to about 0.325; and at least one hydrophobicity additive comprising at least one ceramic nanoparticle, at least one fatty acid, at least one ionic liquid, at least one long chain surfactant, at least one enteric coating, at least one photocurable resin, or combinations thereof.

There is provided, in accordance with an example of the present invention, a surgical adjunct, including a polyurethane foam comprising a volumetric ratio of the polyurethane foam to the total volume of the surgical adjunct is in a range of about 0.125 to about 0.325. The polyurethane foam may include a hydrophobic surface pattern.

There is provided, in accordance with an example of the present invention, a surgical adjunct, including a polyurethane foam including a plurality of pores configured to prevent or reduce fluid ingress into the polyurethane foam.

There is provided, in accordance with an example of the present invention, a surgical adjunct, including a polyurethane foam comprising a volumetric ratio of the polyurethane foam to the total volume of the surgical adjunct is in a range of about 0.125 to about 0.325.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument.

FIG. 2A is a top view of a staple cartridge for use with the surgical stapling and severing instrument of FIG. 1;

FIG. 2B is a side view of the staple cartridge of FIG. 2A;

FIG. 3 is a side view of a staple in an unfired (pre-deployed) configuration that can be disposed within the staple cartridge of the surgical cartridge assembly of FIG. 2A;

FIG. 4 is a perspective view of a knife and firing bar (“E-beam”) of the surgical stapling and severing instrument of FIG. 1;

FIG. 5 is a perspective view of a wedge sled of a staple cartridge of the surgical stapling and severing instrument of FIG. 1;

FIG. 6A is a longitudinal cross-sectional view of an exemplary surgical cartridge assembly having a compressible non-fibrous adjunct attached to a top or deck surface of a staple cartridge;

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct;

FIG. 6C is a perspective view of an exemplary surgical end effector having a channel and a surgical cartridge with a sled and drivers;

FIG. 7 is a partial-schematic illustrating the adjunct of FIGS. 6A-6B in a tissue deployed condition;

FIG. 8A is a perspective view of an exemplary cartridge assembly;

FIG. 8B is a side view of an exemplary adjunct for a cartridge assembly;

FIG. 8C is a top view of an exemplary adjunct for a cartridge assembly;

FIG. 8D is a front view of an exemplary adjunct for a cartridge assembly;

FIG. 8E is a diagram showing an enlarged portion of an exemplary adjunct with a porous structure;

FIG. 8F-1 is a diagram showing an exemplary adjunct for a cartridge assembly with a gradient porosity;

FIG. 8F-2 shows an exemplary surgical adjunct a double gradient of pore diameters;

FIG. 8F-3 shows an exemplary surgical adjunct fabricated from two exemplary surgical adjuncts with each with a gradient of pore diameters;

FIG. 8G is a diagram showing an exemplary adjunct for a cartridge assembly with directional porosity;

FIG. 8H is a diagram showing an exemplary adjunct for a cartridge assembly with directional and closed-cell porosity;

FIG. 8I is a diagram showing an exemplary adjunct for a cartridge assembly with a close cell porosity and hydrophobic film;

FIG. 8J is a diagram showing an exemplary adjunct for a cartridge assembly with a hydrophobic section;

FIG. 8K is a diagram showing an exemplary adjunct for a cartridge assembly with a hydrophobic matrix;

FIG. 8L is a diagram showing an exemplary adjunct for a cartridge assembly with an interlaced hydrophobic material;

FIG. 8M is a diagram showing an exemplary adjunct for a cartridge assembly with hydrophobicity coating;

FIG. 8N is a diagram showing an exemplary adjunct for a cartridge assembly with a patterned surface or portion;

FIG. 9A is a side view of an exemplary adjunct compressed in a dog bone profile;

FIG. 9B is a front view of FIG. 9A.

FIG. 9C is a diagram showing an exemplary adjunct transmurally placed within a patient;

FIG. 9D is a diagram showing an exemplary adjunct transmurally placed within a patient;

FIG. 9E is a diagram showing an exemplary adjunct transmurally placed within a patient; and

FIG. 10 is graph showing the glass transition temperature T_{g} of an exemplary adjunct.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

Surgical stapling assemblies and methods for manufacturing and using the same are provided. In general, a surgical stapling assembly can include a staple cartridge having staples disposed therein and an adjunct configured to be releasably retained on the staple cartridge. As discussed herein, the various adjuncts provided can be configured to compensate for variations in tissue properties, such as variations in tissue thickness, and/or to promote tissue ingrowth when the adjuncts are stapled to tissue.

An exemplary stapling assembly can include a variety of features to facilitate application of a surgical staple, as described herein and illustrated in the drawings. However, a person skilled in the art will appreciate that the stapling assembly can include only some of these features and/or it can include a variety of other features known in the art. The stapling assemblies described herein are merely intended to represent certain exemplary examples. Moreover, while the adjuncts are described in connection with surgical staple cartridge assemblies, the adjuncts can be used in connection with staple reloads that are not cartridge based or any type of surgical instrument.

In transmural procedures, involving the digestive system in particular, there is a risk of the inner fluids wicking through the material and potentially contaminating the body cavity. To eliminate this risk, the surgical adjunct may partially or full block fluid flow by (i) being made to be partially or fully hydrophobic, (ii) including a mechanical barrier within or in addition to the material to block fluid flow, or (iii) including gradient, direction, and/or closed cell (impermeability) morphology. However, completely blocking fluid flow may limit the absorption of the surgical adjunct in the body. In other words, as hydrophobicity or fluid control increases, the associated absorption time increases. Thus, it is important to select the right material, patterns, additives, barriers or morphology to create an adjunct with the desired properties.

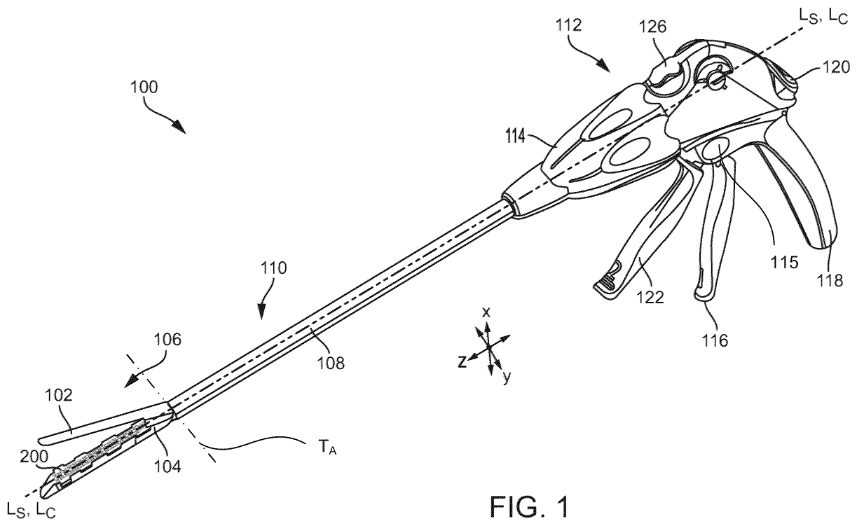

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the staple applying assembly 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108 forming an implement portion 110. When the end effector 106 is closed, or at least substantially closed, (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (TA) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be a powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

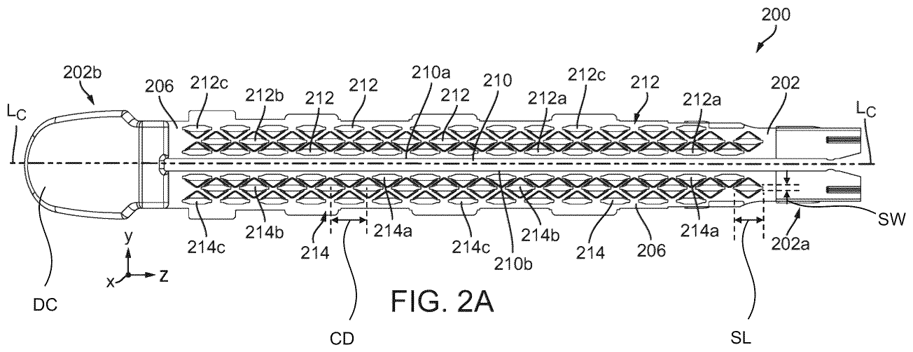

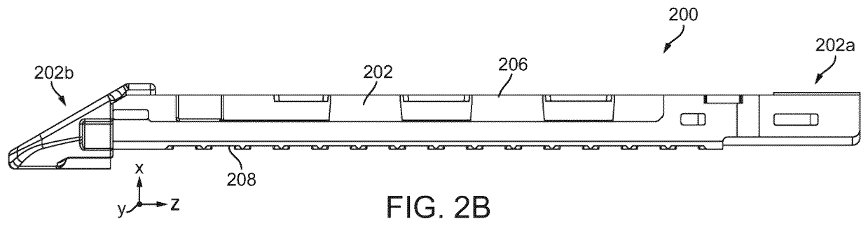

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B, has a proximal end 202a and a distal end 202b with a cartridge longitudinal axis (LC) extending therebetween. As a result, when the staple cartridge 200 is inserted into the elongate channel 104 (FIG. 1), the longitudinal axis (LC) is substantially or approximately parallel with the longitudinal axis (LS) of the elongate shaft 108. Further, the staple cartridge 200 includes a longitudinal slot 210 defined by two opposing walls 210a, 210b and configured to receive at least a portion of a firing member of a firing assembly, like firing assembly 400 in FIG. 4, as discussed further below. As shown, the longitudinal slot 210 extends from the proximal end 202a toward the distal end 202b of the staple cartridge 200. It is also contemplated herein that in other examples, the longitudinal slot 210 can be omitted.

The illustrated staple cartridge 200 includes staple cavities 212, 214 defined therein, in which each staple cavity 212, 214 is configured to removably house at least a portion of a staple (not shown). The number, shape, and position of the staple cavities can vary and can depend at least on the size and shape (e.g., mouth-like shape) of the staples to be removably disposed therein. In this illustrated example, the staple cavities are arranged in two sets of three longitudinal rows, in which the first set of staple cavities 212 is positioned on a first side of the longitudinal slot 210 and the second set of staple cavities 214 is positioned on a second side of the longitudinal slot 210. On each side of the longitudinal slot 210, and thus for each set of rows, a first longitudinal row of staple cavities 212a, 214a extends alongside the longitudinal slot 210, a second row of staple cavities 212b, 214b extends alongside the first row of staple cavities 212a, 214a, and a third row of staple cavities 212c, 214c extends alongside the second row of staple cavities 212b, 214b. Each row may be approximately parallel and the staple cavities that make up the rows may be approximately parallel in orientation with the longitudinal slot 210. As shown in FIGS. 2A, each staple cavity 212, 214 may include a maximum length SL of about 0.122 inches to about 0.124 inches and a maximum width SW of about 0.023 inches to about 0.027 inches. In addition, at least the centers of two adjacent cavities 212, 214 are spaced apart by about 0.158 inches.

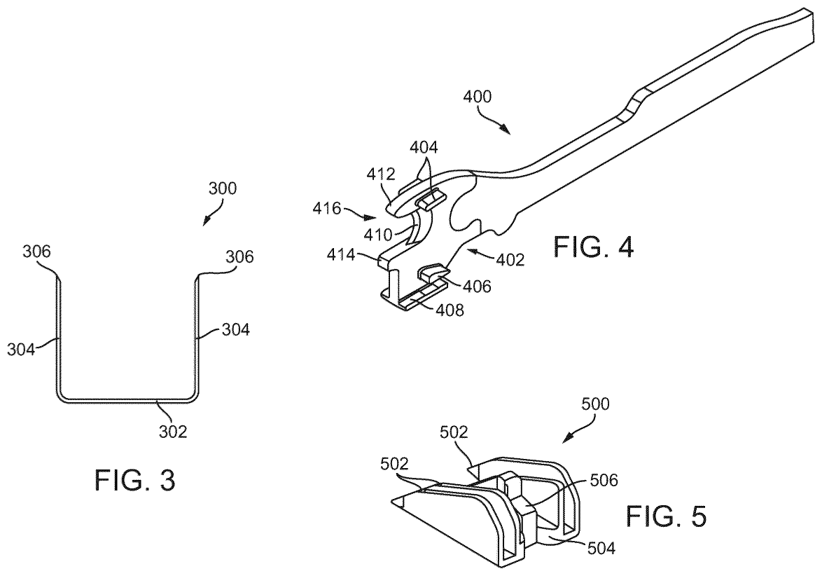

The staples releasably stored in the staple cavities 212, 214 can have a variety of configurations. An exemplary staple 300 that can be releasably stored in each of the staple cavities 212, 214 is illustrated in FIG. 3 in its unfired (pre-deployed, unformed) configuration. The illustrated staple 300 includes a crown (base) 302 and two legs 304 extending from each end of the crown 302. In this example, the crown 302 extends in a linear direction and the staple legs 304 have the same unformed height. Further, prior to the staples 300 being deployed, the staple crowns 302 can be supported by staple drivers that are positioned within the staple cartridge 200 and, concurrently, the staple legs 304 can be at least partially contained within the staple cavities 212, 214. Further, the staple legs 304 can extend beyond a top surface, like top surface 206, of the staple cartridge 200 when the staples 300 are in their unfired positions. In certain instances, as shown in FIG. 3, the tips 306 of the staple legs 304 can be pointed and sharp which can incise and penetrate tissue.

In use, staples 300 can be deformed from an unfired position into a fired position such that the staple legs 304 move through the staple cavities 212, 214, penetrate tissue positioned between the anvil 102 and the staple cartridge 200, and contact the anvil 102. As the staple legs 304 are deformed against the anvil 102, the legs 304 of each staple 300 can capture a portion of the tissue within each staple 300 and apply a compressive force to the tissue. Further, the legs 304 of each staple 300 can be deformed downwardly toward the crown 302 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed, unformed) configuration. In other examples, the staples can include at least two groups of staples each having a different unfired (pre-deployed, unformed) configuration, e.g., varying in height and/or shape, relative to one another, etc.

Referring back to FIGS. 2A-2B, the staple cartridge 200 extends from a top surface or deck surface 206 to a bottom surface 208, in which the top surface 206 is configured as a tissue-facing surface and the bottom surface 208 is configured as a channel-facing surface. As a result, when the staple cartridge 200 is inserted into the elongate channel 104, as shown in FIG. 1, the top surface 206 faces the anvil 102 and the bottom surface 208 (obstructed) faces the elongate channel 104.

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that effects staple formation by the end effector 106.

In use, the anvil 102 in FIG. 1 can be moved into a closed position by depressing the closure trigger in FIG. 1 to advance the E-beam 402 in FIG. 4. The anvil 102 can position tissue against at least the top surface 206 of the staple cartridge 200 in FIGS. 2A-2B. Once the anvil has been suitably positioned, the staples 300 in FIG. 3 disposed within the staple cartridge can be deployed.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be essentially fully absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

### Methods of Stapling Tissue

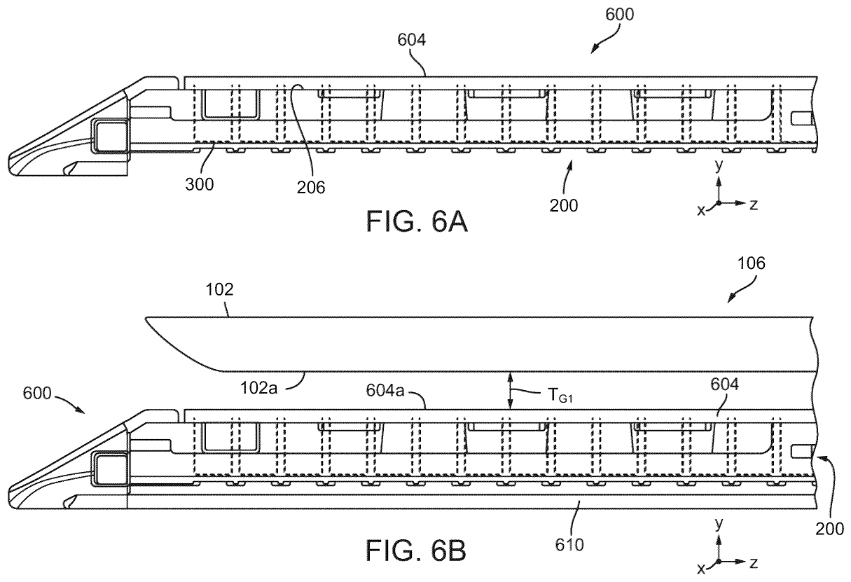

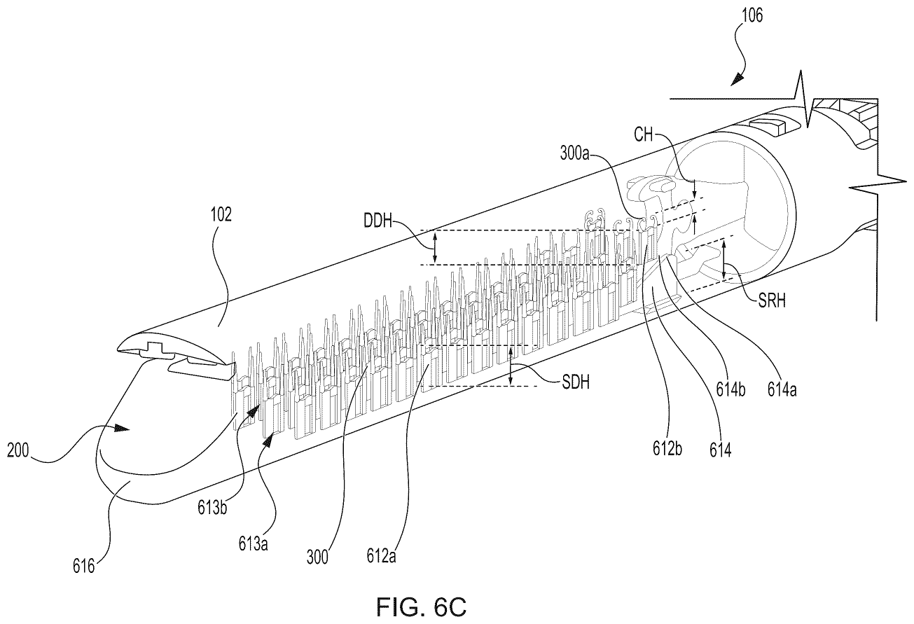

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300, that are configured to be deployed into tissue. The staples 300 can have any suitable unformed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Patent Publication No. 2016/0106427, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap T_{G1} created between the staple cartridge 602 and the anvil 612. More specifically, the tissue gap T_{G1} is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap T_{G1} is generally uniform (e.g., nominally identical within manufacturing tolerances) when no tissue is disposed therein. In other words, the tissue gap T_{G1} is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap T_{G1} can be varied.

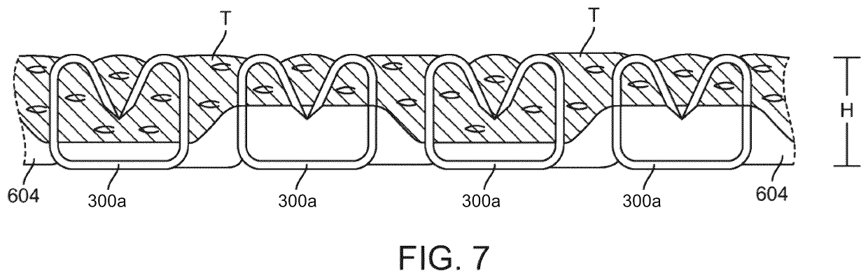

The adjunct 604 is compressible to permit the adjunct to compress to varying heights to thereby compensate for different tissue thickness that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7). That is, the adjunct 604 can have an undeformed state in which a maximum height of the adjunct 604 is greater than a maximum height of a fired staple (e.g., a staple that is in a formed configuration).

As shown in FIG. 6C, the staple cartridge 200 includes a sled 614 and a plurality of drivers 612 configured to drive one or more staples in an upward direction to deploy the staples when a user presses a firing trigger 122 shown in FIG. 1. Once the firing trigger 122 is pressed, the sled 614 moves toward the distal end 616 of the end effector 106 contacting one or more drivers 612a, 612b at a time forcing the one or more drivers 612a, 612b upward along with one or more corresponding staples 300 upward to form fired staple 300a and to capture a material, such as tissue (T), (see FIG. 7.) between the anvil 104 and the fired staple 300a. The cartridge 200 may include a first row 613a of single drivers 612a that corresponds to driving staples 300 positioned in the third row of staple cavities 212c, 214c and a second row 613b of double drivers 612b that corresponds to driving staples 300 positioned in a first row of staple cavities 212a, 214b and a third row 212a, 212b (see FIG. 2A for staple cavities).

One or more single drivers 612a may have a height SDH of about 0.044 inches to about 0.074 inches, such as about 0.050 inches to about 0.068 inches, about 0.054 inches, or about 0.06 inches. One or more double drivers 612b may have a height DDH of about 0.044 inches to about 0.074 inches, such as about 0.050 inches to about 0.068 inches, about 0.054 inches, or about 0.06 inches. The sled 614 may have a least a first rail 614a corresponding to the single drivers 612a positioned in the first row 613a and a second rail 614b corresponding to the double drivers 612b positioned in the second row 613b. The first rail 614a may have a rail height SRH of about 0.164 inches or about 0.167 inches and engages with the single driver 612a. The second rail 614b may have a rail height DRH of about 0.140 inches to about 0.162 inches such as about 0.149 inches or about 0.152 inches and engages with the double driver 612b. Once the staples 300 are deployed, they form a fired staple 300a with a crush height CH of about 0.08 inches to about 0.12 inches such as about 0.97 inches or about 0.1 inches.

In use, once the surgical stapling and severing device, like device 100 in FIG. 1, is directed to the surgical site, tissue is positioned between the anvil 102 and the stapling assembly 600 such that the anvil 102 is positioned adjacent to a first side of the tissue and the stapling assembly 600 is positioned adjacent to a second side of the tissue (e.g., the tissue can be positioned against the tissue-contacting surface 604a of the adjunct 604). Once tissue is positioned between the anvil 102 and the stapling assembly 600, the surgical stapler can be actuated, e.g., as discussed above, to thereby clamp the tissue between the anvil 102 and the stapling assembly 600 (e.g., between the tissue-compression surface 102a of the anvil 102 and the tissue-contacting surface 604a of the adjunct 604) and to deploy staples from the cartridge through the adjunct and into the tissue to staple and attach the adjunct to the tissue.

As shown in FIG. 7, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

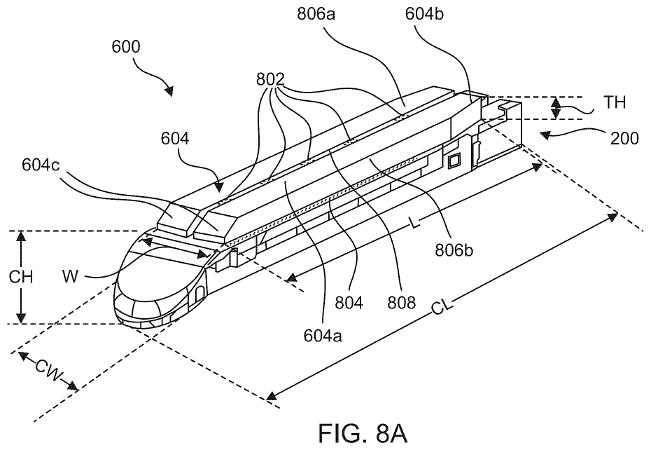

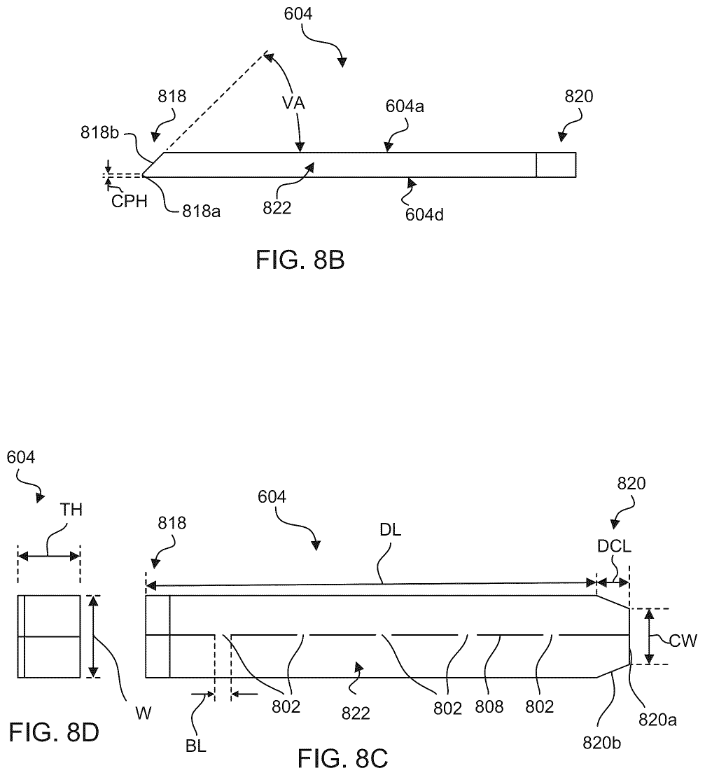

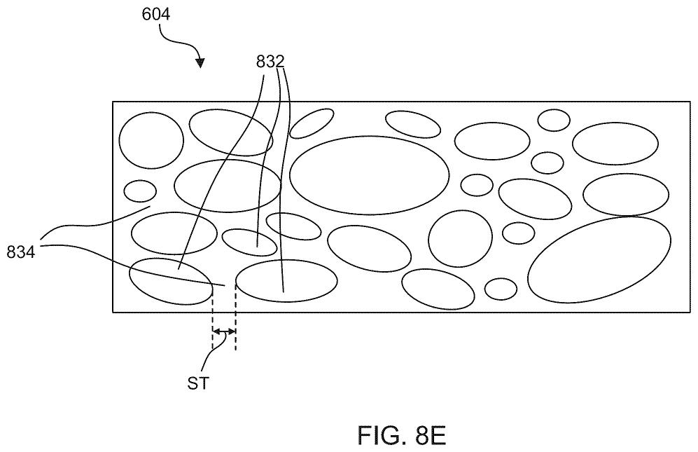

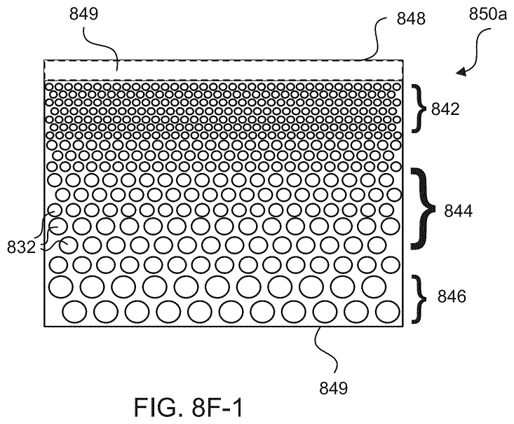

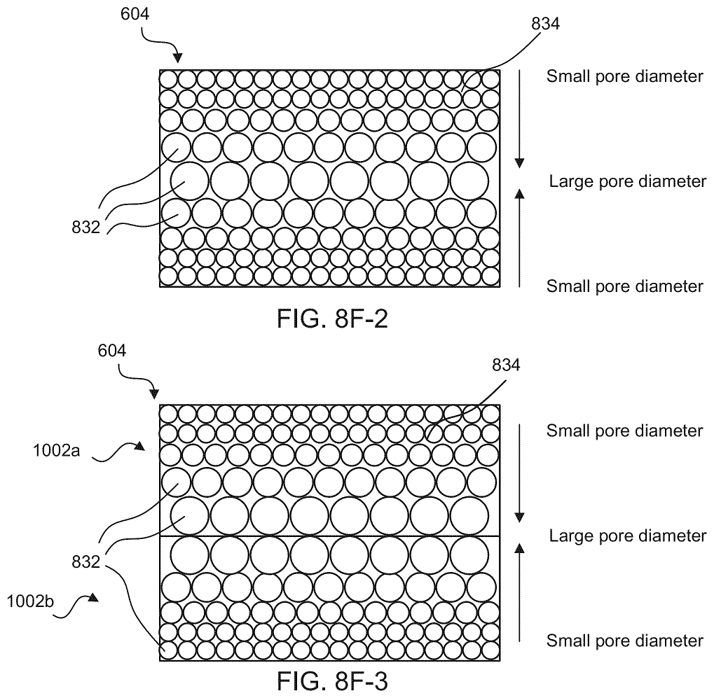

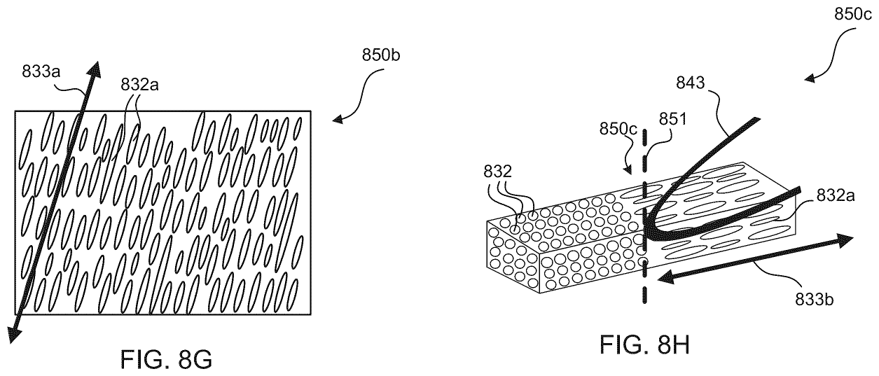

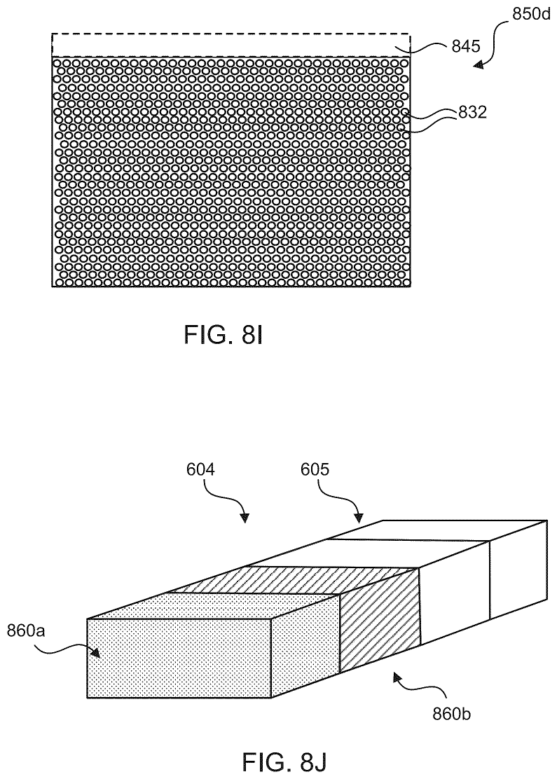

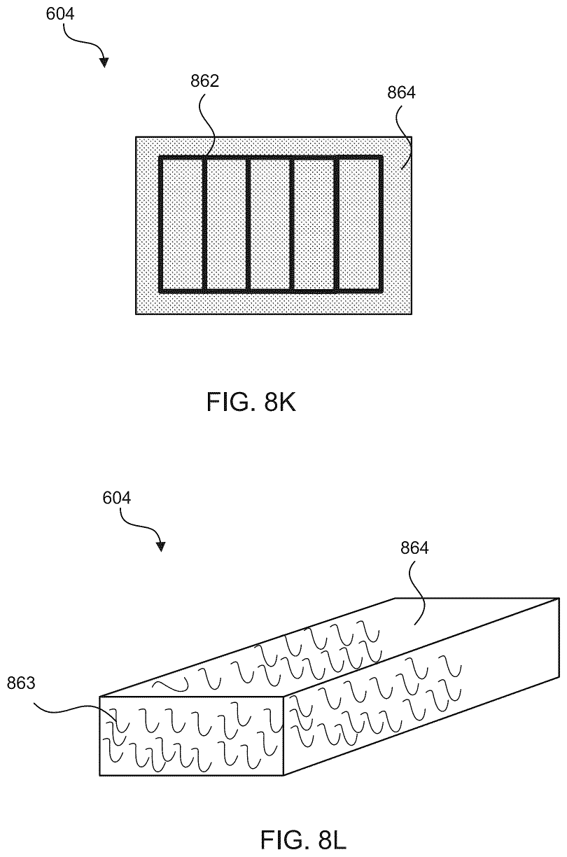

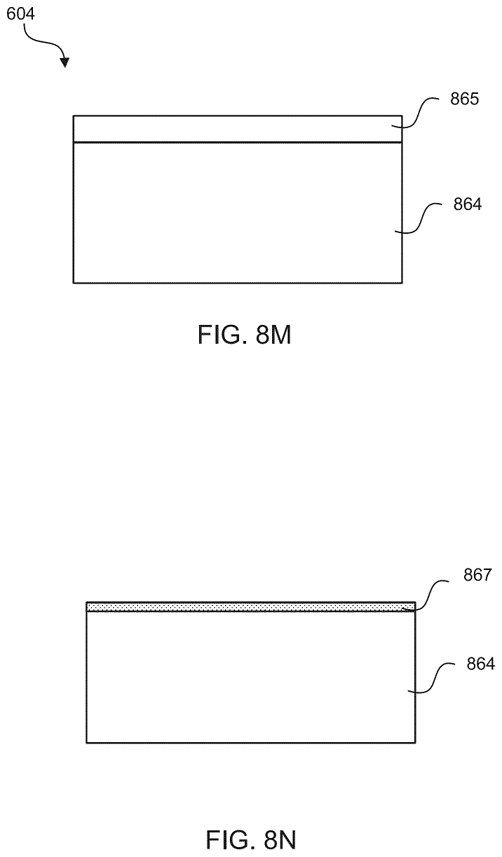

FIG. 8A illustrates a perspective view of a staple cartridge assembly 600 with an adjunct 604 and a staple cartridge 200. The adjunct 604 has a tissue contacting surface 604a, a proximal end 604a, and a distal end 604b. The adjunct 604 may include a slot/slit 808 separating or partially separating two parallel portions of the adjunct 604. In one example, adjunct 604 may include a slot 808 separating two parallel portions of the adjunct 604, while in another example, adjunct 604 may include a slit 808 separating two parallel portions of the adjunct 604 and also one or more bridges (e.g., five bridges) 802 connecting the two parallel portions of the adjunct 604. At least one bridge has a length in the longitudinal direction of about 0.035 inches to about 0.046 inches. The adjunct 604 has a length L of about 40 mm to about 80 mm, such as about 60 mm to about 65 mm, about 66.04 mm to about 66.3 mm, about 45 mm to about 55 mm, or about 51.12 mm to about 51.38 mm. The adjunct 604 has a width W of about 8 mm to about 12 mm, such as about 9.75 mm to about 10.25 mm or about 10.025 mm to about 10.035 mm. The adjunct 604 may also have a thickness or height TH of about 2.5 mm to about 3.5 mm, such as about 2.85 mm to about 3.15 mm or about 2.95 mm to about 3.05 mm.

The cartridge 200 has a height CH of about 6.3 mm to about 8.1 mm, a width CW of about 8.9 mm to about 14 mm, and a length CL of about 80 to about 90 mm such as about 86.7 mm.

Referring to FIGS. 8B-8D, the adjunct 604 has a lower surface 604d and may have a distal chamfered portion 818, a proximal chamfered portion 820, and a center portion 822. The distal chamfered portion 818 has a vertical portion 818a having a height CPH of about 0.009 inches to about 0.029 inches, such as about 0.019 inches. The distal chamfered portion 818 may have an angled portion 818b proximal the vertical portion 818a. The angled portion 818b has a slope VA of about 30 degrees to about 60 degrees, such as about 45 degrees, measured from the tissue contacting surface 604a.

Referring to FIG. 8C, the distal chamfered portion 818 and the center portion 822 has a combined length DL of about 2.25 inches to about 2.45 inches, such as about 2.35 inches. The proximal chamfered portion 820 has an angled portion 820b with a length DCL of about 0.1 inches to about 0.3 inches. In addition, the proximal chamfered portion 820 has a horizontal portion 820a and an angled portion 820n. The horizontal portion 820a may have a width CW of about 0.27 inches to about 0.29 inches, such as about 0.28 inches.

In some examples, the adjunct 604 includes one or more slits 808 with two or more bridges 802 spaced apart by a bridge length BL of about 0.035 inches to about 0.045 inches such as about 0.04 inches.

Referring back to FIG. 8A, the staple cartridge 200 may include one or more raised ledges 804 along one or more sides of the adjunct 602 to help align the adjunct 604 on the deck of the staple cartridge 200.

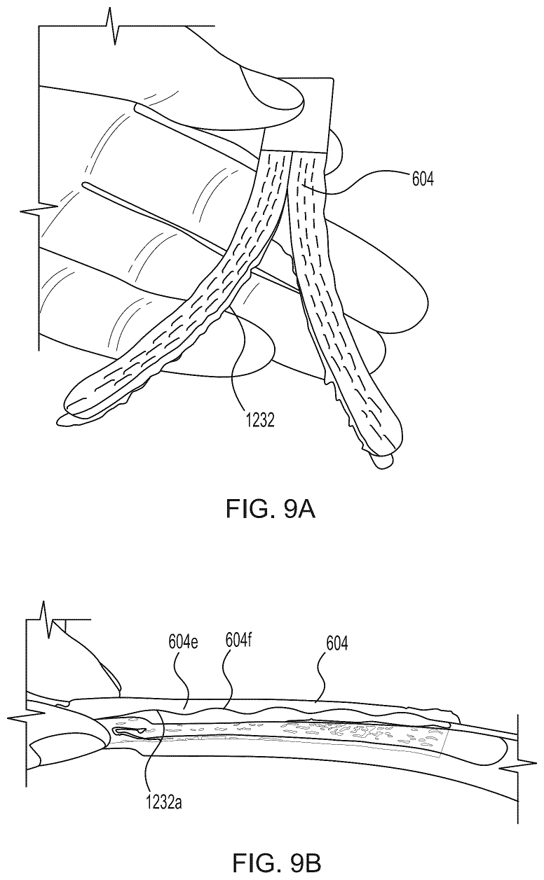

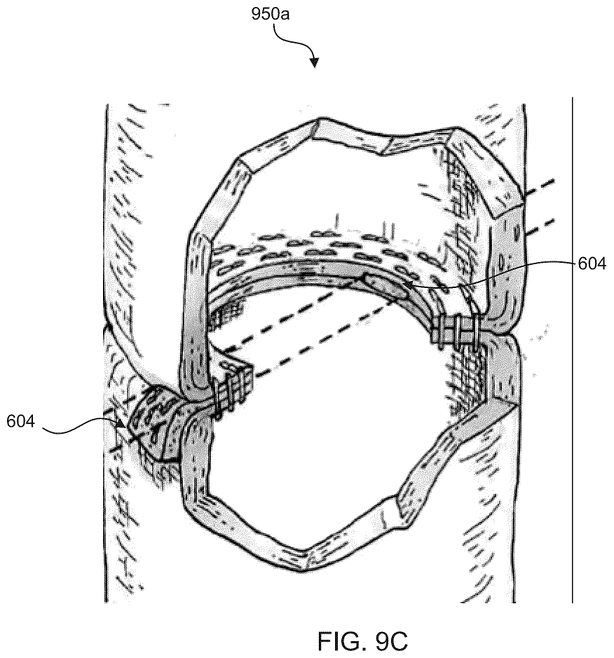

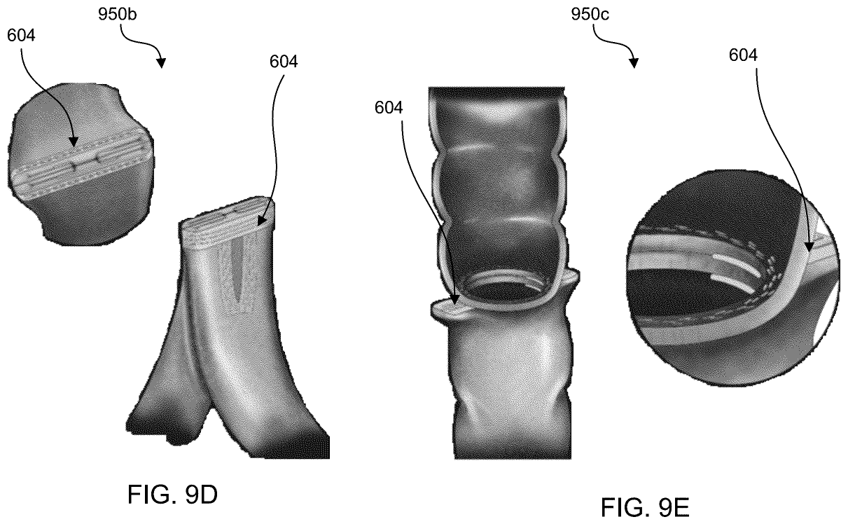

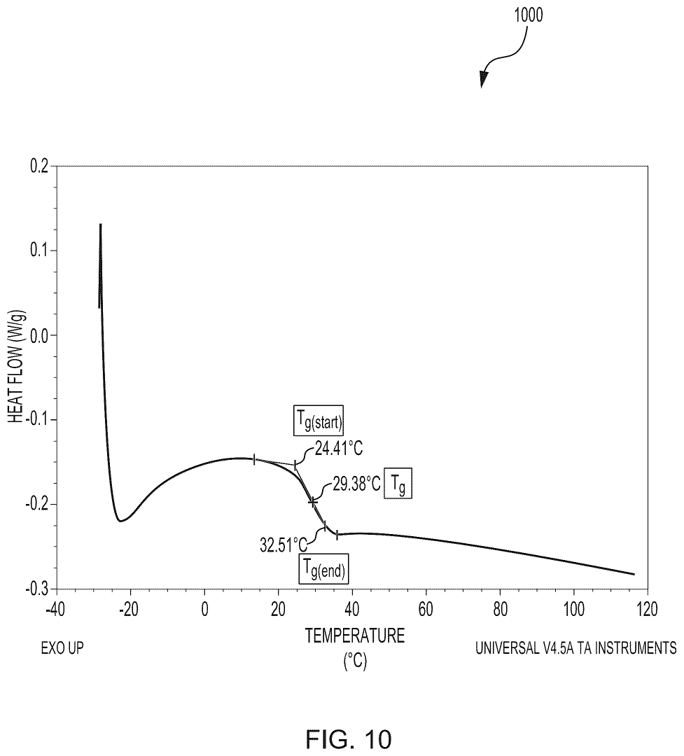

As previously mentioned, the adjunct 604 is compressible. FIGS. 9A and 9B show the dog-bone shape and dimensions that the adjunct 604 takes when subjected to compressibility tests to determine its material properties. As shown, the adjunct 604 has a compression length CPL of about 9.45 mm to about 9.61 mm, such as about 9.53 mm. The adjunct 604 has a compression thickness CT of about 3.15 mm to about 3.21 mm, such as about 3.18 mm. The adjunct 604 has dog bone radius DBR of about 12.62 mm to about 12.78 mm, such as about 12.7 degrees. The adjunct 604 has a dog bone height DBH of about 6.35 mm to about 12.71 mm, such as about 9.53 mm. The adjunct 604 has a dog bone length DBL of about 40 mm to about 80 mm, such as about 63.5 mm. The adjunct has a dog-bone width DBW of about 2.5 mm to about 3.5 mm, such as about 3.03 mm.

Surgical adjunct 604 may be hydrophobic or include a hydrophobic material to prevent fluid ingress from organs such as the colon from contaminating a patient's body cavity. The polyurethane foam may include hydrophobic properties. For example, the polyurethane foam of the surgical adjunct 604 may include a hydrophobic chemical additive when forming the foam. Some examples of additives may include ceramic nanoparticles (e.g., calcium phosphate), fatty acids (e.g., oleic acid, decanoic acid, hexanoic acid, dodecanoic acid), ionic liquids, enteric coating, a photocurable resin, or other long chain surfactants. Fatty acids may have pH dependent solubility. For example, increasing the chain length from hexanoic to decanoic acid can be used to change the solubility in low pHs with increasing solubility in basic conditions. This may be an additional mechanism to include a transient hydrophobicity whereby the fatty acid is entrapped within the network in the presence of acid. The retention would allow for a barrier to fluid flow. In areas not exposed to acidic condition, the fatty acid can be dissolved over a prescribed period to allow for absorption consistent with the neat adjunct material.

The mechanism for ionic liquids would be based on the potential counterion exchange within the body (sodium exchange) to allow dissolution of the liquids within the body. In the associated state, the ionic liquid can provide increased hydrophobicity. As such, the inclusion of ionic liquids can give rise to a transient hydrophobicity. As such, the inclusion of ionic liquids can give rise to a transient hydrophobicity.

As shown in FIG. 8M, the hydrophobic additive may be a hydrophobic coating 865 disposed on the polyurethane foam. A hydrophobic coating 865 would not alter the chemistry of the polyurethane foam and would be layered on top of the foam partially or fully blocking fluid flow. Although the coating is shown on surface it may cover one or more surfaces or a portion thereof. The coating 865 may include multiple layers of different, similar, or the same material to promote coating adhesion of increase hydrophobicity. In addition, multiple layers may be used to minimize impact on adsorption by alternating surface eroding coatings. For example, a surface eroding polyester (e.g., polypropylene fumarate) may be used as the exterior coating with a final later of an enteric coating. The acid generation during the polyester degradation would create a local acidic environment that would eliminate water permeability while retaining a hydrophobic surface. After the acid environment is dissipated, the enteric coating would rapidly dissolve and allow the adjunct to be absorbed. Examples of coating material include at least one fatty acid (e.g., oleic acid, decanoic acid, hexanoic acid, dodecanoic acid), the at least one long chain surfactant, the at least one enteric coating (e.g., cellulose derivatives or methacrylate copolymers), the at least one photocurable resin, or combinations thereof. The hydrophobic coating may be applied to the polyurethane foam via inkjet printing, direct deposition, thermal spraying, cold dynamic spraying, cold spraying, electro spraying, ultrasonic spray coating, dip coating, screen printing, solution deposition, spin coating, film coating, exterior lamination, and lithography.

The surgical adjunct 604 may include multiple different or similar application methods to coat an inner and other surface of the polyurethane foam. The surgical adjunct 604 may include one or more hydrophobic coatings as well as a hydrophobic chemical additive added to the foam. In some examples, the hydrophobic coating may react with a surface of the polyurethane foam to create hydrophobic properties foe the surgical adjunct 604. In some examples, hydrophobic chemical additive and/or the hydrophobic property may include an additional step (e.g., submerging in water, heating, cooling) to activate the hydrophobicity properties.

Furthermore, the hydrophobic coating may be used for time relates therapeutics within the adjunct to assist in healing, reduce inflammation, and treat diseases.

As shown in FIG. 8N, the surgical adjunct 604 may include one or more patterns 867 having one or more hydrophobic surfaces or portions thereof. Hydrophobicity may be generated via specific surface texture on the surface of the surgical adjunct 604. Such surface patterns 867 may have one or more specific repeating patterns with pitches smaller than a diameter of a water droplet. For example, the surface pattern may include at least one repeating pattern with a pitch that is less than about 0.005 centimeters. The specific hydrophobic surface patterns 867 may be generated via interferometric lithography, stereolithography, selective ablation, lyophilization, plasma etching, chemical etching, or surface-initiated polymer brushes.

The advantages of adding one or more of the above-described hydrophobicity features is that such surgical adjunct may be able to mitigate and eliminate transmural waning, mitigate transmural risk in vivo, potentially increase retention of the mechanical strength of the surgical adjunct based on physiological conditions, and potentially use the surgical adjunct 604 as an extended-release intra-organ drug delivery mechanism.

FIGS. 9C, 9D, and 9E are exemplary diagrams 950a, 950b, and 950c showing surgical adjuncts 604 placed transmurally in a patient. For example, biopharmaceuticals such as monoclonal antibodies could be used since it has limited ability to be absorbed within the gastrointestinal tract. The ability to use the surgical adjunct 604 as a carrier for the biopharmaceutical could enable bioavailability within the body without the burden of injections, infusions, etc.

The surgical adjunct 604 may have one or more of the properties described below to enable the adjunct to be flexible when in vivo, but yet remain in a certain position attached to the cartridge when outside of the body. For example, the polyurethane may server to create an adjunct 604 that is flexible when going into the body but “sets” to its final mechanical properties as the plasticizer is absorbed in vivo. In some examples, a plasticizer may be added to the polyurethane foam to lower its glass transition temperature to be within the below described ranges as well as conform to the other listed properties. Regardless, an adjunct 604 having one or more of the below properties consistently creates a hemostatic or near hemostatic seal on tissue.

The surgical adjunct 604 may include a polyurethane foam with or without a plasticizer where the glass transition temperature of the surgical adjunct 604 is about 0° C. to about 40° C. (e.g., about 19.4° C.), such as about 7.5° C. to about 22.5° C. or about 12.5° C. to about 17.5° C. The glass transition temperature of the adjunct 604 is obtained by using a standard differential scanning calorimetry (DSC) system. Using the DSC system with its output shown in FIG. 13, an adjunct 604 was equilibrated at about −40° C., heated at about 40° C./min to about 120° C., held isothermally for about 1 minute, cooled at about 40° C./min to about −40° C., held isothermally for about 1 minute, and then heated at about 10° C./min to about 120° C., where the glass transition temperature T_{g} was measured and recorded by the DSC system.

The surgical adjunct 604 may include a volumetric ratio of the polyurethane foam to the total volume of the adjunct 604 of about 0.125 to about 0.325, such as about 0.175 to about 0.225 or about 0.19 to about 0.21. The total volume may include air (from pores of the foam) or other material beside the foam structures.

The plasticizer may include one or more of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid. In some examples, the plasticizer includes one or more fatty acids.

Referring to FIG. 8E, the adjunct 604 may include a polyurethane foam with pores 832 having a median pore size of about 0.025 mm³ to about 0.300 mm³, such as about 0.022 mm³. In some examples, the adjunct 604 may have one or more struts 834 between the pore 832 that provide support and strength to the adjunct 604. In particular, the adjunct 604 may include a plurality of struts 834, having a median strut thickness ST of about 0.025 mm to about 0.300 mm, such as about 0.08 mm.

In some examples, the adjunct 604 includes a polydioxanone (PDO) film disposed on one or more surfaces of the polyurethane foam. In some examples, the PDO film is adhered to at least a bottom or crown side of the adjunct 604. In some examples, the PDO film has a thickness of about 20 μm to about 100 μm, such as about 40 μm.

The adjunct 604 may have a compression strength of about 30 kPa to about 70 kPa, such as about 30 kPa to about 60 kPa (e.g., about 42 kPa), about 30 kPa to about 50 kPa, about 32.5 kPa to about 37.5 kPa. In order to test compression strengths, an adjunct 604 was placed in a humid warm environment at approximately 37° C., compressed to a first height, then a second height shorter than the first height, and then released back to the first height at which point the adjunct's compression strength was measured.

In some examples, the adjunct 604 may have a tensile strength of about 30 kPa to about 90 kPa such as about 45 kPa to about 85 kPa or about 55 kPa to about 75 kPa. In some examples, the adjunct 604 will have tensile strength of about 110 kPa to about 150 kPa. Tensile strength is measured on an adjunct 604 having the dog-bone configuration shown and described with respect to FIGS. 9A and 9B. Specifically, the adjunct's 604 tensile strength is measured after submerging it in water at a temperature of about 37° C. for less than a minute and then running a tensile strength test.

The manipulation of the foam morphology of the surgical adjunct 604 to prevent transmural pathways could have significant impacts on the versatility of the foam's applications in the medical market. Through manipulation of the morphology, whether it be gradient, directional, or closed-cell/impermeable, the risk of transmural infections can be greatly reduced while maintaining the overall purpose and biocompatibility of the surgical adjunct 604.

Referring to FIG. 8F-1, the surgical adjunct 604 may include a polyurethane foam with a gradient porosity 850a with pores 832 including small pores 842 disposed adjacent to a first end or first side 848. When placed in a patient's body, the first end 848 may be positioned adjacent to a point of transmural access from a patient's organ inside the patient's body cavity. The pores 832 of polyurethane foam may also include large pores 846 disposed away from the first end 848 and adjunct a second end or second side 849. The polyurethane foam may also include transitional pores 844 that include pores generally arranges such that smaller diameters pores are closer to the first end 848 and larger diameter pores are positioned further away from first end 848. The small pores 842 and some of the transitional pores 844 (e.g., the smaller ones) may prevent the fluid ingress from the patient's organ (e.g., organ from digestive system) into the surgical adjunct the rest of the patient's body while the large pores 846 as well as some of the transitional pores 844 (e.g., some of the larger ones) may allow fluid ingress from other parts of the body such as the blood stream to promote biodegradation of the polyurethane foam. Although pores 832 are described are increasing in size from the first end 848 as they travel away from the first end 848, the pores 832 may increase in size along any direction such as the axial direction 851 (FIG. 8H), lateral direction 833b (FIG. 8H), or both.

Referring to FIG. 8F-2, the surgical adjunct 604 may include a double density gradient with respect to its pore diameters. For example, the surgical adjunct 604 may include pores 832 with small diameters adjacent a first end and a second end that is opposite the first end. Moving from the first end or second end to the center of the surgical adjunct 604 the diameters of the pores 832 continue to increase to a large diameter size. Referring to FIG. 8F-3, the surgical adjunct 604 may include two bioabsorbable materials 1002a, 1002b, each with a single gradient for pore diameters. These two bioabsorbable materials 1002a, 1002b may be joined together to form a double density gradient. In both FIGS. 8F-2 and 8F-3, the surgical adjunct may include struts 834 that may increase in thickness near the larger pores than the smaller pores.

Referring to FIGS. 8G and 8H, the surgical adjunct 604 may include a polyurethane foam with directional morphology. For example, the pores 832a of the polyurethane foam may be aligned in a first direction 833a to generally control fluid flow in the first direction 833a and second direction 833a (opposite directional arrow), which is a direction opposite the first direction (see FIG. 8G). As another example, the polyurethane foam may include directional pores 832a aligned in a first direction 833b (also lateral direction) as well as include small pores 832 or closed cells pores (see FIG. 8H). Both the directional pores 832a and the small pores 832 (or closed cell pores) may prevent fluid 843 through certain portions of the polyurethane foam but allow fluid 843 to flow through the portion of the foam that includes the directional pores.

Using a thin impermeable skin would prevent fluid transport through the cushion while being able to maintain optimal porosity within the surgical adjunct 604. Additionally, through reduction of surfactant content, the foam can be manipulated to achieve a closed-cell morphology with thin membranes surrounding the cells. This would prevent moisture wicking through the porous structure for a determined period of time while ethe tissue heals. Once the tissue is healed. The membranes would slowly breakdown allowing full cell penetration and tissue ingrowth into the cushion for biodegradation.

Referring to FIG. 8I, the surgical adjunct 604 may include an impermeable barrier, skin, or moisture barrier 845 disposed on one or more surfaces of the polyurethane foam. The polyurethane foam may include small pores (or closed cells pores) 832.

Referring to FIG. 8J, the surgical adjunct 604 may include a section of impermeable or hydrophobic material 860b or a moisture barrier along with a buttress material (e.g., an adhesive) 860a section and one or more sections of polyurethane foam 605. The hydrophobic material 860b is disposed between the buttress material 860 and the polyurethane foam 605. The moisture barrier may include a film including aliphatic polyesters such as polylactic acid (PLA), polyglycolic acid (PGA), polycaprolactone (PCL), polydioxanone (PDO), poly(trimethylene carbonate) (PTMC), or copolymer combinations.

Referring to FIG. 8K, the surgical adjunct 604 may include matrix 862 of a hydrophobic film or moisture barrier within the polyurethane foam 864. Referring to FIG. 8L, the surgical adjunct 604 may include material 863 interlaced within the polyurethane foam 864. The material 863 may be include a hydrophobic coating or be hydrophobic. The moisture barrier may include a film including aliphatic polyesters such as polylactic acid (PLA), polyglycolic acid (PGA), polycaprolactone (PCL), polydioxanone (PDO), poly(trimethylene carbonate) (PTMC), or copolymer combinations.

As will be appreciated by one skilled in the art, The embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

In some examples, polyurethane foam is used as a major component of the surgical adjunct 604. Other bioabsorbable materials may be used and are envisioned.

In some examples, disclosed devices (e.g., end effector, surgical adjunct, and/or staple cartridges) and methods involving one or more disclosed devices may involve one or more of the following clauses:

Clause 1: A surgical adjunct 604, comprising: a polyurethane foam comprising a volumetric ratio of the polyurethane foam to the total volume of the surgical adjunct 604 is in a range of about 0.125 to about 0.325; and at least one hydrophobicity additive comprising at least one ceramic nanoparticle, at least one fatty acid, at least one ionic liquid, at least one long chain surfactant, at least one enteric coating, at least one photocurable resin, or combinations thereof.

Clause 2: The surgical adjunct 604 of Clause 1, wherein the at least one hydrophobicity additive comprises the at least one fatty acid comprising oleic acid, decanoic acid, hexanoic acid, dodecanoic acid, or combinations thereof.

Clause 3: The surgical adjunct 604 of Clause 2, wherein the at least one fatty acid comprises oleic acid.

Clause 4: The surgical adjunct 604 of Clause 1, wherein the at least one hydrophobicity additive comprises the at least one ceramic nanoparticle.

Clause 5: The surgical adjunct 604 of Clause 4, wherein the at least one ceramic nanoparticle comprises calcium phosphate.

Clause 6: The surgical adjunct 604 of Clause 1, wherein the at least one hydrophobicity additive comprises the at least one long chain surfactant.

Clause 7: The surgical adjunct 604 of Clause 1, wherein the at least one hydrophobicity additive is a hydrophobic coating disposed on the polyurethane foam.

Clause 8: The surgical adjunct 604 of Clause 7, wherein the hydrophobic coating comprises the at least one fatty acid, the at least one long chain surfactant, the at least one enteric coating, the at least one photocurable resin, or combinations thereof.

Clause 9: A surgical adjunct 604, comprising: a polyurethane foam comprising a volumetric ratio of the polyurethane foam to the total volume of the surgical adjunct 604 is in a range of about 0.125 to about 0.325, wherein the polyurethane foam comprises a hydrophobic surface pattern.

Clause 10: The surgical adjunct 604 of Clause 9, wherein the surface pattern comprises at least one repeating pattern with a pitch that is less than about 0.005 centimeters.

Clause 11: A surgical adjunct 604, comprising: a polyurethane foam comprising a volumetric ratio of the polyurethane foam to the total volume of the surgical adjunct 604 is in a range of about 0.125 to about 0.325; and a moisture barrier dividing the polyurethane foam into at least two sections and configured to prevent fluid ingress.

Clause 12: The surgical adjunct 604 of Clause 11, wherein the moisture barrier comprises a film comprising an aliphatic polyester.

Clause 13: The surgical adjunct 604 of Clause 11, wherein the moisture barrier comprises a matrix splitting the polyurethane foam.

Clause 14: The surgical adjunct 604 of Clause 11, wherein the moisture barrier comprises one or more poly(p-dioxanone) coated sutures interlaced within the polyurethane foam.

Clause 15: A surgical adjunct 604, comprising: a polyurethane foam comprising a plurality of pores configured to prevent or reduce fluid ingress into the polyurethane foam.

Clause 16: The surgical adjunct 604 of Clause 15, wherein a volumetric ratio of the polyurethane foam to the total volume of the surgical adjunct 604 is in a range of about 0.125 to about 0.325.

Clause 17: The surgical adjunct 604 of Clause 15, wherein the plurality of pores form a gradient or double gradient based on an average diameter and with respect to a first side of the polyurethane foam

Clause 18: The surgical adjunct 604 of Clause 15, wherein at least some of the plurality of pores are aligned in a first direction to direct fluid ingress within the polyurethane foam at least in a first direction and a second direction opposite the first direction.

Clause 19: The surgical adjunct 604 of Clause 18, wherein at least some of the plurality of pores are close celled pores configured to prevent fluid ingress.

Clause 20: The surgical adjunct 604 of Clause 15, wherein at least some of the plurality of pores are closed cell pores configured to prevent fluid ingress.

Clause 21: A surgical adjunct 604, comprising: a polyurethane foam comprising a volumetric ratio of the polyurethane foam to the total volume of the surgical adjunct 604 is in a range of about 0.125 to about 0.325.

Clause 22: The surgical adjunct 604 of Clause 21, further comprising: at least one hydrophobicity additive comprising at least one ceramic nanoparticle, at least one fatty acid, at least one ionic liquid, at least one long chain surfactant, at least one enteric coating, at least one photocurable resin, or combinations thereof.

Clause 23: The surgical adjunct 604 of Clause 22, wherein the at least one hydrophobicity additive comprises the at least one fatty acid comprising oleic acid, decanoic acid, hexanoic acid, dodecanoic acid, or combinations thereof.

Clause 24: The surgical adjunct 604 of Clause 23, wherein the at least one fatty acid comprises oleic acid, decanoic acid, hexanoic acid, dodecanoic acid, or combinations thereof.

Clause 25: The surgical adjunct 604 of Clauses 22 to 24, wherein the at least one hydrophobicity additive comprises the at least one ceramic nanoparticle.

Clause 26: The surgical adjunct 604 of Clauses 22 to 25, wherein the at least one ceramic nanoparticle comprises calcium phosphate.

Clause 27: The surgical adjunct 604 of Clauses 22 to 26, wherein the at least one hydrophobicity additive is a hydrophobic coating disposed on the polyurethane foam, the hydrophobic coating comprising the at least one fatty acid, the at least one long chain surfactant, the at least one enteric coating, the at least one photocurable resin, or combinations thereof.

Clause 28: The surgical adjunct 604 of Clause 21 to 27, wherein the polyurethane foam comprises a hydrophobic surface pattern.

Clause 29: The surgical adjunct 604 of Clause 28, wherein the surface pattern comprises at least one repeating pattern with a pitch that is less than about 0.005 centimeters.

Clause 30: The surgical adjunct 604 of Clauses 21 to 29 further comprising a moisture barrier dividing the polyurethane foam into at least two sections and configured to prevent fluid ingress.

Clause 31: The surgical adjunct 604 of Clause 30, wherein the moisture barrier comprises a film comprising an aliphatic polyester.

Clause 32: The surgical adjunct 604 of Clauses 30 or 31, wherein the moisture barrier comprises a matrix splitting the polyurethane foam.

Clause 33: The surgical adjunct 604 of Clauses 30 to 32, wherein the moisture barrier comprises one or more poly(p-dioxanone) coated sutures interlaced within the polyurethane foam.

Clause 34: The surgical adjunct 604 of Clauses 21 to 33, wherein the polyurethane foam comprises a plurality of pores configured to prevent or reduce fluid ingress into the polyurethane foam.

Clause 35: The surgical adjunct 604 of Clause 34, wherein the plurality of pores (i) have an average diameter of the plurality of pores that form a gradient or double gradient with respect to a first side of the polyurethane foam, (ii) are aligned in one direction to direct fluid ingress within the polyurethane foam, (iii) comprise closed cell pores, or combinations thereof.

1-15. (canceled)

16. A surgical adjunct, comprising:
a polyurethane foam comprising a volumetric ratio of the polyurethane foam to the total volume of the surgical adjunct is in a range of about 0.125 to about 0.325; and
at least one hydrophobicity additive comprising at least one ceramic nanoparticle, at least one fatty acid, at least one ionic liquid, at least one long chain surfactant, at least one enteric coating, at least one photocurable resin, or combinations thereof.

17. The surgical adjunct of claim 16, wherein the at least one hydrophobicity additive comprises the at least one fatty acid comprising oleic acid, decanoic acid, hexanoic acid, dodecanoic acid, or combinations thereof.

18. The surgical adjunct of claim 17, wherein the at least one fatty acid comprises oleic acid.

19. The surgical adjunct of claim 16, wherein the at least one hydrophobicity additive comprises the at least one ceramic nanoparticle.

20. The surgical adjunct of claim 19, wherein the at least one ceramic nanoparticle comprises calcium phosphate.

21. The surgical adjunct of claim 16, wherein the at least one hydrophobicity additive comprises the at least one long chain surfactant.

22. The surgical adjunct of claim 16, wherein the at least one hydrophobicity additive is a hydrophobic coating disposed on the polyurethane foam.

23. The surgical adjunct of claim 22, wherein the hydrophobic coating comprises the at least one fatty acid, the at least one long chain surfactant, the at least one enteric coating, the at least one photocurable resin, or combinations thereof.

24. A surgical adjunct, comprising: a polyurethane foam comprising a volumetric ratio of the polyurethane foam to the total volume of the surgical adjunct is in a range of about 0.125 to about 0.325, wherein the polyurethane foam comprises a hydrophobic surface pattern.

25. The surgical adjunct of claim 24, wherein the surface pattern comprises at least one repeating pattern with a pitch that is less than about 0.005 centimeters.

26. A surgical adjunct, comprising: a polyurethane foam comprising a volumetric ratio of the polyurethane foam to the total volume of the surgical adjunct is in a range of about 0.125 to about 0.325; and a moisture barrier dividing the polyurethane foam into at least two sections and configured to prevent fluid ingress.

27. The surgical adjunct of claim 26, wherein the moisture barrier comprises a film comprising an aliphatic polyester.

28. The surgical adjunct of claim 26, wherein the moisture barrier comprises a matrix splitting the polyurethane foam.

29. The surgical adjunct of claim 26, wherein the moisture barrier comprises one or more poly(p-dioxanone) coated sutures interlaced within the polyurethane foam.

30. A surgical adjunct, comprising: a polyurethane foam comprising a plurality of pores configured to prevent or reduce fluid ingress into the polyurethane foam.

31. The surgical adjunct of claim 30, wherein a volumetric ratio of the polyurethane foam to the total volume of the surgical adjunct is in a range of about 0.125 to about 0.325.

32. The surgical adjunct of claim 30, wherein the plurality of pores form a gradient or double gradient based on an average diameter and with respect to a first side of the polyurethane foam.

33. The surgical adjunct of claim 30, wherein at least some of the plurality of pores are aligned in a first direction to direct fluid ingress within the polyurethane foam at least in a first direction and a second direction opposite the first direction.

34. The surgical adjunct of claim 33, wherein at least some of the plurality of pores are close celled pores configured to prevent fluid ingress.

35. The surgical adjunct of claim 30, wherein at least some of the plurality of pores are closed cell pores configured to prevent fluid ingress.
